(19) United States
(12) Patent Application Publication
(10) Pub. No.: US 2017/0112923 A1
(43) Pub. Date: Apr. 27, 2017

(54) MULTI-VALENT ADJUVANT DISPLAY

(71) Applicant: Psioxus Therapeutics Limited, London (GB)

(72) Inventors: Leonard Charles William Seymour, Oxfordshire (GB); Kerry Fisher, Gloucester (GB)

(21) Appl. No.: 15/299,936

(22) Filed: Oct. 21, 2016

Related U.S. Application Data
(63) Continuation of application No. 13/318,844, filed on Feb. 21, 2012.

(30) Foreign Application Priority Data
May 8, 2009 (GB) 0907989.8

(51) Int. Cl. A61K 39/39; A61K 39/145; C12N 7/00; A61K 39/00

(52) U.S. Cl. CPC A61K 39/39; A61K 47/48215; A61K 39/0011; A61K 47/48176; A61K 47/48784; A61K 47/48061; A61K 39/145; C12N 7/00; A61K 47/48023; A61K 2039/55511; C12N 2760/16022; C12N 2760/16034

(57) ABSTRACT

The present invention provides an adjuvant-polymer construct comprising a polymer backbone which is covalently linked to 3 or more adjuvants, wherein the 3 or more adjuvants are each present in a pendant side chain, the adjuvants being connected to the polymer backbone either directly or via a spacer group.

### CROSS REFERENCE TO RELATED APPLICATIONS

This Application is a Continuation application which claims priority to U.S. Non-provisional application Ser. No. 13/318,844, filed Feb. 21, 2012, which is a §371 National Stage Application of International PCT Application PCT/GB2010/000915, filed on May 7, 2010, which claims priority to Foreign Application GB0907989.8, filed May 8, 2009, all of which are incorporated herein by reference in their entirety.

The present invention relates to improved techniques for stimulating the immune system using adjuvants. The invention particularly relates to new adjuvant-containing products which present adjuvants in a multiple display format, compositions including these products and the use of these products in immunisation.

### BACKGROUND

Adjuvants are typically components (or analogues) of common pathogens such as viruses, bacteria or fungi. They are normally recognised by pattern receptors, scavenging receptors and toll-like receptors (TLRs). Most successful adjuvants bind to these receptors with low affinity but high avidity due to multiple-repeat presentation. The best adjuvants tend to be whole (or partially degraded) bacteria or double stranded viral DNA. However, there is currently a move in the art towards more defined formulations with a single identifiable, and preferably fully synthetic, component. Unfortunately clean, discrete and mono-dispersed adjuvants do not stimulate the innate immune system to the same extent as the original ‘dirty’ formulations. Contemporary synthetic adjuvants such as imiquimod and Pam2Cys are poorly soluble low molecular weight agents that are difficult to formulate and deliver.

Particular examples of adjuvants described in the art include synthetic adjuvants such as those described in U.S. Pat. No. 6,149,222. These adjuvants are poloxamers made up of polyoxyethylene/polyoxypropylene block copolymers and they stimulate a variety of cell surface receptors by a poorly defined non-ionic interaction. U.S. Pat. No. 6,610,310 describes poly-anionic synthetic polymers made up of multiple negative charges on a synthetic sugar or other polymer. Such synthetic adjuvants, however, generally have poor avidity and insufficient adjuvant activity.

Efforts have been made to incorporate synthetic adjuvants into formulations that aim to improve delivery. For example, US 2005/0233105 describes formulations that include a low molecular weight synthetic adjuvant. However, these formulations are simple mixtures of adjuvants with a viral vaccine and they do not provide a means to improve the intrinsic activity of the synthetic adjuvants.

Similarly, WO 2007/078879 describes compositions comprising self-assembling liposomes, polymer complexes and emulsified lipids. These compositions are intended to present adjuvants in a more natural format, but they are difficult to formulate and much of the adjuvant material is inaccessible as it is trapped within the hydrophobic core. These formulations congeal under certain conditions and due to their instability they normally have to be made up immediately prior to administration.

There is therefore a need for a new approach in the design of synthetic adjuvants which provide improved stimulation of the immune system.

### SUMMARY OF THE INVENTION

The present invention therefore provides an adjuvant-polymer construct (also referred to herein as a polymer-adjuvant construct) comprising a polymer backbone which is covalently linked to 3 or more adjuvants, wherein the 3 or more adjuvants are each present in a pendant side chain, the adjuvants being connected to the polymer backbone either directly or via a spacer group.

The present inventors have found that linking several small synthetic adjuvants to a polymer so that the adjuvants are presented in a multi-valent display format can increase immune stimulation compared to the use of the synthetic adjuvants alone. The presentation of multiple adjuvants in this way, reminiscent of pathogen-associated molecular patterns (PAMPs), is thought to improve receptor avidity and to provide a more natural presentation to toll-like receptors and pattern recognition receptors. Furthermore, the multi-valent display of the adjuvants encourages receptor cross-linking and signalling. These factors all lead to increased immune stimulation and thereby enable lower doses of adjuvant to be used and side effects to be decreased.

Linking adjuvants to a polymer chain in this way also increases the molecular size of the adjuvant component which helps to prevent leaching into the blood stream and thereby to reduce off-target toxicity.

In preferred embodiments of the invention the polymer backbone itself is hydrophilic which helps to solubilise the typically lipophilic adjuvantss. This facilitates the delivery of the adjuvant and enables much simpler formulations to be used. A further advantage is the increase in the number of molecules which can interact with the receptors, also enabling lower doses to be used.

The adjuvant-polymer construct of the invention is typically administered in conjunction with a vaccine. The present invention therefore also provides a vaccine conjugate comprising an adjuvant-polymer construct of the invention which is bound to a vaccine. Also provided is a composition comprising an adjuvant-polymer construct or vaccine conjugate of the invention and a pharmaceutically acceptable carrier or diluent.

The present invention also provides a method for stimulating or enhancing an immune response in a subject in need thereof, comprising administering to said subject an effective, non-toxic amount of an adjuvant-polymer construct, vaccine conjugate or composition of the invention. When the adjuvant-polymer construct or composition does not comprise a vaccine, the method further comprises the step of administering a vaccine, either simultaneously or separately. Also provided is an adjuvant-polymer construct, vaccine conjugate or composition of the invention, for use in a method of stimulating or enhancing an immune response.

### BRIEF DESCRIPTION OF THE FIGURES

FIGS. 1a-1d provide a scheme showing the synthesis of a ceramide analogue.

FIG. 2 depicts the structure of a polymer-adjuvant construct according to the invention.

FIG. 3a depicts the structure of a reactive polymer for use in preparing a polymer-adjuvant construct. FIG. 3b depicts the same polymer bound to a ceramide adjuvant and a peptide antigen.

FIG. 4 shows multiple presentation of the TLR 2 agonist Pam3Cys on the same polymer results in higher stimulation of cells. U937 is a lymphoma cell line with monocyte-like phenotype that expresses a range of TLR receptors including TLR2. Stimulation of TLR2 results in activation of NfkB and these cells have been transfected with a reporter plasmid (luciferase) under the control of the NfkB promoter. After an 8 hour exposure to the test substance, the cells were lysed and analysed for luciferase expression. All test substances were added at 100 ng/ml (note only 5.22 ng of the Pam3Cys polymer conjugate is Pam3Cys). The data shows that polymer bound Pam3Cys shows significantly higher level of stimulation relative to Pam3Cys on its own and comparable to the positive control LPS.

FIG. 5 shows multiple presentation of the TLR 2 agonist Pam2Cys on the same polymer results in higher stimulation of cells. Bone marrow derived DCs were exposed to 50 ng/ml Pam2Cys (P2C) or P2C linked to HPMA for 24 hours. The cells supernatant was then evaluated for IL-8 by ELISA. Note only 5 wt % (2.5 ng) of Pam2Cys was present in the 50 ng/ml sample of P2C-pHPMA.

### DETAILED DESCRIPTION OF THE INVENTION

An adjuvant as used herein is an agent that may stimulate the immune system and increase the response to a vaccine, without having any specific antigenic effect in itself.

The constructs of the present invention contain at least 3 adjuvants, which may be the same or different. In one preferred embodiment, the 3 or more adjuvants are the same. Each adjuvant is bound to the polymer backbone in a pendant side chain. The adjuvants are therefore not a part of the polymeric backbone itself. This provides a better presentation of the adjuvants for recognition by cellular receptors, since the spatially-associated array of adjuvants more closely resembles ‘TAMP’ epitopes on the surface of a whole bacterium or virus, or a component thereof such as double stranded viral RNA, and leads to greater binding avidity for the cellular receptor or receptors.

At least 3 adjuvants, preferably at least 5, more preferably at least 10 adjuvants, are covalently bound to each polymer, each adjuvant being present typically on a separate pendant side chain. Typically up to 50 adjuvants are present on a single polymer backbone. In one embodiment at least 20 adjuvants are present on the polymer backbone.

Adjuvants may comprise a broad range of structures, characterised by their ability to promote an improved immune response against a vaccine. One class of receptors to which adjuvants bind are the Toll-like receptors (TLRs; also known as ‘pattern receptors’, because of their ability to recognise repeated PAMP sequences on the surface of pathogens). TLRs recognize conserved molecular products derived from various classes of pathogens, including Gram-positive and -negative bacteria, DNA and RNA viruses, fungi and protozoa. TLR genes have been recognized in a number of vertebrate genomes, and many partial and full-length sequences are available. Eleven TLRs have been identified in humans while 13 can be found in searches of the mouse genome. Human and mouse TLR family members have been shown to have distinct ligand specifities, recognizing different molecular structures. TLR1, TLR2, TLR4, TLR5 and TLR6 are all localized to the plasma membrane recognizing cell wall components, while TLR3, TLR7, TLR8 and TLR9 are preferentially expressed in intracellular compartments such as endosomes and recognize nucleic acid structures. The ligand requirements of different TLRs have been partially characterised:

TLR2 heterodimers (mainly with TLR1 or TLR6)—lipoproteins, peptidoglycans, lipoglycans, lipoteichoic acid, lipopolysaccharide, peptidoglycans, zymosan

TLR5—bacterial flagellins

TLR7—imidazoquinolines (Imiquimod, GArdiquimod), CL264, Loxoribine

TLR8—single stranded RNA, E coli RNA

TLR7/TLR8 heterdimers: CL075, CL097, Poly(dT), R848

TLR9—unmethylated CpG islands in DNA, including CpG-containing oligonucleotides

TLR4—lipopolysaccharide, monophosphoryl lipid A

TLR3—double stranded RNA

Examples of TLR ligands include:
Imiquimod—ligand for TLR7
MALP-2 (a diacylated lipopeptide isolated from Mycoplasma Fermentans)—ligand for TLR6/TLR2
Porphyromonas gingivalis lipopolysaccharide, lipoteichoic acid—ligands for TLR2
Pam3CSK4—ligand for TLR2 heterodimer with TLR1
Pam2Cys—ligand for TLR2 heterodimer with TLR6
Poly(I).Poly(C)—ligand for TLR3

TLRs are found in innate immune cells (DCs, macrophages, natural killer cells), cells of the adaptive immunity (T and B lymphocytes) and also in non immune cells (epithelial and endothelial cells, fibroblasts).

Preferred adjuvants for use in the invention are lipoglycans, lipopolysaccharide, lipoarabinomannan, lipoteichoic acid, peptidoglycan, natural and synthetic lipoproteins and lipopeptides, zymosan, glycolipids, Polyinosine-polycytidylic acid, monophosphoryl Lipid A, TNFα, TNF peptides, CD40 ligand, OX40, IL-4, IL-6, IL-8, IL-2, IL-12, mannose, GM-CSF, IFN-gamma, IFN-alpha, Flagellin, imidazoquinoline-compounds, guanosine, double-stranded RNA (dsRNA), single-stranded DNA (ssDNA) and unmethylated CpG-containing sequences in bacterial DNA or synthetic oligonucleotides.

The polymeric backbone of the present invention may be a synthetic or naturally occurring polymer. A polymer as used herein includes biopolymers such as nucleic acids, proteins and starch as well as synthetic polymers. In one embodiment, the polymer is not a nucleic acid. In another embodiment, the polymer is a synthetic polymer.

In one embodiment the polymers are hydrophilic polymers which will impart water solubility to the adjuvant-polymer construct. For example, the adjuvant-polymer constructs may have a water solubility of at least 100 μg/mL, for instance at least 150 μg/mL or at least 200 μg/mL.

The polymer itself may be biologically active, for example the polymer may itself have adjuvant properties. In one embodiment, the polymer itself has substantially no adjuvant activity (i.e. the polymer alone would not increase immune stimulation). Measurement of the adjuvant activity of the polymer may be carried out, for example, using the popliteal lymph node (PLN) assay, where the polymer is injected into the hind footpad of mice together with an antigen. Adjuvant activity is determined as the increase in PLN weight and cell numbers in animals receiving antigen together with the substance under study, compared with PLN weight and cell numbers in animals given the antigen without the substance in question, and animals given the putative adjuvant alone. In another embodiment, the polymer is biologically inactive.

Suitable synthetic polymers include those based on monomers having a vinyl moiety, for example (meth)acrylates, (meth)acrylamides, vinyl, vinyl ether, vinyl ester and styryl moieties. Particular examples of monomers in this category are (meth)acrylates and (meth)acrylamides, in particular N-2-hydroxypropylmethacrylamide (HPMA) and hydroxyethylmethacrylate (HEMA), and vinylpyrrolidone (PVP). Cyclic monomers suitable for ring-opening polymerisation and ring-opening metathesis can also be used, for example cyclic amides, cyclic esters, cyclic urethanes, cyclic ethers, cyclic anhydrides, cyclic sulfides, cyclic amines and mono- and multi-cyclic alkenes. Alternative polymer backbones include nucleic acids (e.g. polyI:polyC, polyA:polyU, single stranded DNA, double stranded DNA), polyethylene glycol, poly(ethylene glycol-oligopeptide), poly(amino acids) (eg poly[N-(2-hydroxyethyl)-L-glutamine) and polysaccharides such as glycogen, cellulose, dextran, cyclodextrin, alginate, hyaluronic acid, polysialic acid, polymannan or other polymers based on glucose or galactose. Further natural products which can be used as the polymer backbone include heparin, dextran and starch. Where the backbone is based upon ethyleneglycol-oligopeptide, the oligopeptide group preferably comprises from 1 to 4 amino acids and the pendant side chains are typically supported by the oligopeptide portion of the polymer backbone.

Typically, the polymer backbone is based on monomer units chosen from (meth)acrylates, (meth)acrylamides, styryl monomers, vinyl monomers, vinyl ether monomers, vinyl ester monomers, sialic acid monomers, mannose monomers, N-(2-hydroxyethyl)-L-glutamine (HEG) monomers, and ethyleneglycol-oligopeptide monomers. Preferably, the polymer backbone is based on monomer units chosen from N-2-hydroxypropylmethacrylamide (HPMA), N-(2-hydroxyethyl)-L-glutamine (HEG), and ethyleneglycol-oligopeptide, or is a polysialic acid or polymannan polymer.

Polymer backbones based on HPMA are more preferred.

The weight average molecular weight of the polymer is typically in the region of from 1 to 100 kDa, for example at least 2, more preferably at least 5 kDa and up to 80 kDa, more preferably up to 40 kDa. Preferred polymers have a weight average molecular weight of from 5 to 40 kDa.

The polymer backbone may be a linear polymer having 3 or more pendant side chains comprising an adjuvant. Alternatively, the polymer backbone itself may be a branched structure. For example, dendritic and comb polymers are envisaged.

In some embodiments, the polymer backbone may be cross-linked to further polymers such that it forms a hydrogel. The hydrogel is preferably hydrolytically unstable or is degradable by an enzyme, for example matrix metalloproteinases 2 or 9. This is in order that the adjuvants are immobilised within the hydrogel and so that the release of the adjuvants can be regulated. Thus, according to one preferred feature of the invention, the process of the invention is carried out under conditions likely to promote crosslinking and hydrogel formation (for example high concentrations of reagents with none present in excess) or in the presence of agents such as diamines likely to promote crosslinking. Formation of hydrogels containing modified adjuvants would generally be performed using the chemical approaches described in Subr, V., Duncan, R. and Kopeck, J. (1990) “Release of macromolecules and daunomycin from hydrophilic gels containing enzymatically degradable bonds”, J. Biomater. Sci. Polymer Edn., 1 (4) 61-278.

Where the polymer backbone comprises a nucleic acid, the polymer may be linked to a further nucleic acid to form a double stranded helical structure.

The adjuvants may be connected to the polymer backbone either directly or via a spacer group. In a preferred embodiment, a spacer group is present, such that the adjuvant-polymer construct has the structure:

P[S-A]ₙ

where P is the polymer backbone, S is a spacer group, A is an adjuvant and n is 3 or more.

The spacer groups may be the same or different and are typically selected from oligo(alkyloxide)s (e.g. pEG chains which are from 2 to 200 carbon atoms in length); oligopeptides having, for example, up to about 20 amino acids; C1-C12 alkyl moieties (e.g. C1-C6 alkyl moieties such as methylene, ethylene, propylene or butylene); C6-C10 aryl moieties (e.g. phenyl); combinations of such alkyl and aryl moieties; polyesters and polycarbonates. Suitable polyesters and polycarbonates are, for example, those having chains of from 10 to 30 carbon atoms. The spacer group is typically hydrophilic and may incorporate a degradable linkage such as a reducible disulphide bond, a bond susceptible to acid-catalysed hydrolysis or a bond cleavable by enzymatic degradation.

Preferred spacer groups are oligo(alkoxide)s and oligopeptides, in particular oligopeptides.

In one embodiment of the invention, the spacer group is an oligopeptide. Preferably, the oligopeptide contains up to 10, for example up to 5 amino acids. More preferably, the oligopeptide contains from 1 to 4, for example 2 or 4 amino acids. Suitable oligopeptides are -Gly-Phe-Leu-Gly-, -Gly-Gly- and Glu-Lys-Glu-.

In another embodiment the spacer group incorporates a degradable linkage. For example, the spacer group may be cleavable by reduction, for example: —NH—(CH₂)₂NHCO—(CH₂)₂—SS—(CH₂)₂—CO—. Alternatively, the spacer group may be cleavable by acid-catalysed hydrolysis, for example:

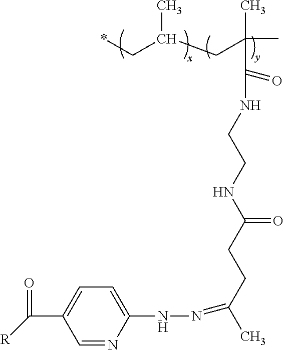

where x and y are independently integers of from 1 to 5, e.g. 1, 2 or 3 and R is, for example, a C1-C8 alkyl group e.g. methyl or ethyl.

The value of n reflects the number of pendant side chains which comprise an adjuvant. At least 3 adjuvants are present on each polymer molecule, so n is at least 3. Typically up to 50 adjuvants are present on a single polymer backbone, so n is up to 50. In one embodiment at least 20 adjuvants are present on the polymer backbone.

In one embodiment of the invention, the groups -S-A comprise at least 2 mol % of the adjuvant-polymer construct. For example, the groups -S-A may comprise at least 5 mol % of the construct. Typically, the groups -S-A comprise no more than 20 mol % of the adjuvant-polymer, for example up to 10 mol %.

The adjuvant-polymer construct of the invention may contain pendant side chains bearing functional groups other than adjuvants. Such functional groups may be bound directly to the polymer backbone or via a spacer group. Suitable spacer groups are those described above. Examples of functional groups which may be present are solubilising groups such as amine, hydroxyl, carboxyl and oligo(alkylene) groups.

Examples of adjuvant-polymer constructs of the invention are those of formula P-[S-A]ₙ, wherein P is a polymer based on monomer units chosen from N-2-hydroxypropylmethacrylamide (HPMA), N-(2-hydroxyethyl)-1-glutamine (HEG), and ethyleneglycol-oligopeptide, or is a polysialic acid or polymannan polymer; S is -Gly-Phe-Leu-Gly-, -Gly-Gly- or Glu-Lys-Glu-; n is from 3 to 10; and A is an adjuvant as defined above.

Several different approaches to the synthesis of the constructs of the invention are envisaged:
1. Copolymerisation of simple monomers with functionalised monomers to generate a polymer backbone having reactive groups on pendant side chains, followed by attachment of adjuvantss to these reactive groups.
2. Functionalisation of the adjuvantss, or adjuvant-spacer molecules, to incorporate polymerisable groups, and addition of the functionalised adjuvant or adjuvant-spacer to the polymerisation mixture.
3. Adaptation of a natural or synthetic polymer by binding adjuvantss either directly or via a spacer group.

In the case of a synthetically-produced polymer, suitable polymerisation techniques include free radical polymerisation techniques such as conventional and controlled techniques, e.g. NMP (nitroxide mediated radical polymerisation), ATRP (Atom Transfer Radical Polymerisation), RAFT (Reversible addition—fragmentation chain transfer) or cyanoxyl-based polymerisation, for example as described in Scales, C. W.; Vasilieva, Y. A.; Convertine, A. J.; Lowe, A. B.; McCormick, C. L. Biomacromolecules 2005, 6, 1846-1850; Yanjarappa, M. J.; Gujraty, K. V.; Joshi, A.; Saraph, A.; Kane, R. S. Biomacromolecules 2006, 7, 1665-1670; Convertine, A. J.; Ayres, N.; Scales, C. W.; Lowe, A. B.; McCormick, C. L. Biomacromolecules 2004, 5, 1177-1180, the entirety of which are incorporated herein by reference. Cyclic monomers can be polymerised using ring-opening polymerisation or ring-opening metathesis.

Relevant teaching can also be found in ‘Macromolecular design via reversible addition-fragmentation chain transfer (RAFT)/xanthates (MADIX) polymerization.’ Perrier, Sebastien; Takolpuckdee, Pittaya. J. Polym. Sci., Part A: Polym. Chem. (2005), 43(22), 5347-5393, which is incorporated herein by reference.

Typically an initiator is used in the copolymerisation reaction, preferably AIBN. The reaction generally takes place in an organic solvent, typically DMSO. The reaction is usually heated to a temperature of from 50 to 70° C., preferably about 60° C. The reaction is usually heated to the above-specified temperature for from 4 to 8 hours, preferably 5 to 7 hours, more preferably about 6 hours. The thus-obtained polymers are typically precipitated in an acetone-diethyl ether (3:1) mixture, filtered off, washed with acetone and diethyl ether and dried in vacuo. The thus-obtained polymers may be further purified in Sephadex-LH 20 columns using methanol.

The monomers for the polymerisation reaction are typically commercially available or may be prepared by analogy with known methods, for example as described in Konak, et al, Langmuir, 2008, 24, 7092-7098.

Synthesis (1) described above involves the inclusion of functionalised monomers in the polymerisation reaction. Such functionalised monomers typically have the structure PG-S-F or PG-F, wherein PG is a polymerisable monomer such as HPMA or methacrylamide (suitable monomers are further defined above), S is a spacer group as defined above and F is a functional group. Mixtures of two or more different functionalised monomers may be used, if desired.

In this case, the polymerisation mixture will include both non-functionalised monomers and functionalised monomers. The functionalised monomers are typically incorporated into the polymer chain in an amount of up to about 20 mol %, for example up to 10 mol %. Preferably, the functionalised monomer is incorporated in an amount of at least 2 mol %, for example at least 5 mol %.

Suitable functional groups F include solubilising groups such as those described above, and reactive groups. Protected groups which are precursors to such solubilising or reactive groups may also be used.

It will be understood that the term “reactive group” is used herein to denote a group that shows significant chemical reactivity, especially in relation to coupling or linking reactions with complementary reactive groups of other molecules, typically with groups on the adjuvant.

Reactive groups can be used as the point of attachment of an adjuvant or vaccine or an alternative functional group such as a solubilising group. For example, the reactive group may be capable of forming a covalent bond with, for example, an amine group, thiol group, hydroxy group, aldehyde, ketone, carboxylic acid or sugar group on the adjuvant, vaccine or other molecule containing an alternative functional group. In the case of reaction with an adjuvant or vaccine, the adjuvant or vaccine may be functionalised, if necessary, to include such a group capable of forming a covalent bond with a reactive group.

In one embodiment, the reactive group is capable of forming a covalent bond with an amine group. Examples of suitable types of reactive group in this embodiment include acid chlorides, isocyanates, isothiocyanates, acyl-thiazolidine-2-thiones, maleimides, N-hydroxy-succinimide esters (NHS esters) sulfo-N-hydroxy-succinimide esters (Sulfo-NHS esters), 4-nitrophenol esters, epoxides, 2-imino-2-methoxyethyl-1-thioglycosides, cyanuric chlorides, imidazolyl formates, succinimidyl succinates, succinimidyl glutarates, acyl azides, acyl nitriles, dichlorotriazines, 2,4,5-trichlorophenols, azlactones and chloroformates. Such groups react readily with amines. Acyl-thiazolidine-2-thiones and Sulfo-NHS esters are preferred. Acyl-thiazolidine-2-thiones are preferred due to their high reactivity and relative stability in aqueous solutions.

In another embodiment, the reactive group is capable of forming a covalent bond with a thiol group. Examples of suitable types of reactive group in this embodiment include alkyl halides, haloacetamides, and maleimides.

In another embodiment, the reactive group is capable of forming a covalent bond with a hydroxyl group. Examples of suitable types of reactive group in this embodiment include chloroformates and acid halides. Alternatively, hydroxyl groups can be oxidised with an oxidizing agent, e.g. periodate, followed by reaction with reactive groups that include hydrazines, hydroxylamines or amines.

In another embodiment, the reactive group is capable of forming a covalent bond with an aldehyde or ketone group. Examples of suitable types of reactive group in this embodiment include hydrazides, semicarbazides, primary aliphatic amines, aromatic amines and carbohydrazides.

In another embodiment, the reactive group is capable of forming a covalent bond with a carboxylic acid. This can be effected by, for example, activating a carboxylic acid using the water soluble carbodiimide, 1-ethyl-3-(3-dimethylaminopropyl)carbodiimide hydrochloride followed by reaction with an amine as reactive group.

In another embodiment, the reactive group is capable of reacting with a sugar to form a covalent bond. This can be effected by, for example, enzyme-mediated oxidation of the sugar with galactose oxidase to form an aldehyde followed by reaction with an aldehyde reactive compound such as a hydrazide as reactive group.

Preferred examples of reactive groups are nitrophenol esters (ONp), N-hydroxysuccinimide (NHS), thiazolidine-2-thione (TT) and epoxy groups.

Following polymerisation, the reactive groups incorporated into the polymer may be directly reacted with adjuvants or converted to other functionalities such as solubilising groups. Alternatively, the reactive groups may be partially reacted with a molecule containing an alternative reactive group leading two the presence of two different reactive functionalities. These reactive groups can then be further modified using two orthogonal methods.

Examples of suitable polymers containing functionalised pendant side chains are disclosed in WO 98/19710 and include polyHPMA-GlyPheLeuGly-ONp, polyHPMA-GlyPheLeuGly-NHS, polyHPMA-Gly-Gly-ONp, polyHPMA-Gly-Gly-NHS, poly(pEG-oligopeptide(-ONp)), poly(pEG-GluLysGlu(ONp)), pHEG-ONp, pHEG-NHS. The preparation of these compounds is disclosed in WO 98/19710. The contents of WO 98/19710 is included herein by reference. Another such functionalised polymer suitable for use in the invention is polyHPMA-GlyPheLeuGly-TT (where TT is thiazolidine-2-thione), the synthesis of which is described in WO 2005/007798. The contents of WO 2005/007798 in included herein by reference.

Alternative methodologies for synthesis of the constructs of the invention involve the functionalisation of an adjuvant for inclusion in the polymerisation mixture (synthesis (2) above). In this embodiment, the polymerisation is typically carried out as described above, but using a functionalised monomer having the structure PG-S-A or PG-A, wherein PG and S are as defined above and A is the adjuvant. Mixtures of two or more such functionalised monomers, or mixtures of such functionalised monomers with those of formula PG-S-F or PG-F as described above, may be used.

As described above, the functionalised monomers are incorporated in an amount of up to about 20 mol %, for example up to 10 mol %. Preferably, the functionalised monomer is incorporated in an amount of at least 2 mol %, for example at least 5 mol %.

In a further embodiment, the construct of the invention is obtained by adaptation of a pre-formed polymer, such as a naturally occurring polymer (synthesis (3) above). In this case, suitable reactive groups on the polymer are used for addition of the adjuvants, optionally via a spacer group, and any further desired functional groups such as solubilising groups.

In one embodiment of the invention, the polymer backbone contains two or more different adjuvants. In this embodiment, the adjuvants may be randomly arranged or in a particular sequence. For example, the polymer may comprise a block copolymer of structure -A-B-A-B- wherein A is a polymer section having one or more pendant side chains including an adjuvant (a) and B is a polymer section having one or more pendant side chains including an adjuvant (b). Such selected sequences of adjuvants may provide a synergistic effect.

In a further embodiment of the invention, the polymer backbone and/or the spacer groups on the pendant side chains are degradable. Degradable linkages may therefore be present in the polymer backbone and/or within one or more pendant side chains. Degradable linkages are those which can break down in vivo, either spontaneously or through a specifically triggered event. Typically, degradable linkages are tailored for spontaneous hydrolysis following endosomal uptake by the falling endosomal pH, or may be linkages which are reducibly cleaved in the intracellular reducing environment. Alternatively, degradable linkages may be designed for cleavage by particular enzymes.

The use of biodegradable linkages is advantageous to promote degradation of the polymer-adjuvant conjugate, restricting its adjuvant activity and facilitating eventual excretion to avoid unwanted toxicity.

Some polymers for use in the present invention are inherently degradable, for example some nucleic acids. Alternatively, degradable linkages may be incorporated into the polymer backbone or side chains. Examples of such degradable linkages include disulphide bonds, which are typically cleaved using mild reducing conditions, such as a metal sulfite or a suitably chosen enzyme; hydrazone bonds, cis-aconityl bonds and ortho esters that are cleaved by pH dependent hydrolysis; or bonds that are enzymatically cleavable.

Enzymatically cleavable bonds are designed for cleavage by particular enzymes and typically involve short oligopeptides such as the oligopeptides described herein as spacer groups.

Instability provided by enzymatic degradability can be desirable since it permits the polymer (or the linkage between the polymer and the adjuvant) to be designed for cleavage selectively by chosen enzymes. Such enzymes could be present at the target site, endowing the modified adjuvant with the possibility of triggered disintegration at the target site, thereby releasing the adjuvant for interaction with the target tissue. The enzymes may also be intracellular enzymes which can bring about disintegration of the modified adjuvant in selected cellular compartments of a target cell to enhance the activity of the adjuvant. Alternatively, enzyme-cleavage sites may be designed to promote disintegration of the modified adjuvant in response to appropriate biological activity (eg. arrival of an invading or metastatic tumour cell expressing metalloproteinase). In a further variation, enzymes capable of activating the modified adjuvant may be administered at the appropriate time or site to mediate required disintegration of the modified adjuvant and subsequent interaction of the adjuvant with the tissue.

The adjuvant-polymer constructs of the invention are suitable for administration to a human or mammalian subject in conjunction with a vaccine, to enhance or stimulate the immune response of that patient to the vaccine.

Examples of suitable vaccines which can be used with the present invention include viruses, proteins, peptides, sugars and nucleic acids. Vaccines may be prophylactic (given to protect the recipient from disease) or therapeutic (to assist the immune system in attacking an existing infection or disorder). In general vaccines may be dead or inactivated organisms, purified products derived from them, synthetic peptides, recombinant proteins or nucleic acid vaccines that encode components of the target organism.

Some vaccines contain killed microorganisms—these are previously virulent micro-organisms which have been killed with chemicals or heat. Examples are vaccines against flu, cholera, bubonic plague, and hepatitis A.

Attenuated vaccines contain live, attenuated virus microorganisms—these are live micro-organisms that have been engineered or cultivated under conditions that disable their virulent properties, or which use closely-related but less dangerous organisms to produce a broad immune response. They typically provoke more durable immunological responses and are the preferred type for healthy adults. Examples include yellow fever, measles, rubella, and mumps. The live tuberculosis vaccine is not the contagious strain, but a related strain called “BCG”; it is used in the United States very infrequently.

Further examples of vaccine classes are:

Toxoids—these are inactivated toxic compounds in cases where these (rather than the micro-organism itself) cause illness. Examples of toxoid-based vaccines include tetanus and diphtheria. Not all toxoids are vaccines for micro-organisms; for example, Crotalis atrox toxoid is used to vaccinate dogs against rattlesnake bites.

Peptide vaccines—synthetic peptides containing antigenic epitopes from disease proteins for example influenza M2e peptide. In principle, any peptide can be incorporated onto the adjuvant containing polymer either singularly or in multiple copies (1-20). Preferred peptides contain no critical lysine residue in the sequence other than that which is added to enable conjugation to the polymer. For peptides containing lysine residues in the active site, alternative conjugation through the side chain of cysteine residues may be used.

Protein subunit vaccines—rather than introducing an inactivated or attenuated micro-organism to an immune system (which would constitute a “whole-agent” vaccine), a fragment of it can create an immune response. Characteristic examples include the subunit vaccine against Hepatitis B virus that is composed of only the surface proteins of the virus (produced in yeast) and the virus-like particle (VLP) vaccine against human papillomavirus (HPV) that is composed of the viral major capsid protein.

Conjugate vaccines—certain bacteria have polysaccharide outer coats that are poorly immunogenic. By linking these outer coats to proteins (e.g. toxins), the immune system can be led to recognize the polysaccharide as if it were a protein antigen. This approach is used in the Haemophilus influenzae type B vaccine.

Recombinant vaccines are where a vector (sometimes an innocuous virus, sometimes a plasmid) is used as a ‘Trojan Horse’ to introduce and express genes encoding components of the target pathogen within the cells of the recipient, including within antigen-presenting cells such as dendritic cells. For example attenuated adenovirus vectors may be used to express proteins from target pathogens (eg. components of malaria, tuberculosis, influenza) within host cells, enabling production of an immune response against the target pathogen without exposing the recipient to any infectious material. Similar approaches can be used for a variety of viral vectors, notably alpha virus.

In one embodiment, the vaccine is conjugated to the adjuvant-polymer construct to form a vaccine conjugate. A very broad range of vaccines may be conjugated in this way, including peptide, lipid, protein, nucleic acid, carbohydrate and synthetic vaccines, including vaccines of mixed composition. The vaccines may be derived from many targets, including viruses, protozoa, nematodes, fungi, yeasts or bacteria, or they may be intended to vaccinate against cancer-associated antigens. The linkage between vaccine and polymer-adjuvant conjugate may be designed for degradation following association with cells, to enhance the cellular trafficking of the vaccine component. Such biodegradable linkages may be reducible linkages, hydrolytically unstable linkages of linkages that are substrates for degradation by target-associated enzymes.

The range of possible vaccines includes, but is clearly not restricted to:
Influenza peptides and influenza proteins, for example those used in standard influenza vaccines including H and N proteins and M2.
Peptides derived from HIV, including epitopes from gp120, gp41, gag, nef and pol.
HepB surface antigen
Cancer antigens such as CEA, MUC-1 and 5T4
Anthrax proteins, subunits and peptides
Norovirus proteins and peptides
Toxoids (see above)
Proteins, subunits and peptide antigens from diverse strains of plague (Yersinia pestis)
Proteins and peptides derived from malaria—for example the circumsporozoite antigen or synthetic TRAP epitope string
Viruses and Virus-Like-Particles (VLPs) including adenovirus, respiratory syncytial virus, alphavirus, herpesvirus, vaccinia virus, measles, reovirus and lentiviruses.

The conjugation of polymer-adjuvant construct and vaccine is achieved by providing one or more reactive groups on the polymer-adjuvant construct which are capable of binding to groups on the vaccine. Binding may be covalent or by another type of interaction such as electrostatic attraction. Typically, more than one reactive group, e.g. at least 5 reactive groups, are provided so that several connections between the vaccine and the polymer-adjuvant construct are formed.

In the case of covalent bonding between the polymer-adjuvant construct and the vaccine, the reactive groups described in detail above may be used. The exact nature of the reactive group will depend on the available binding sites on the vaccine. Examples of preferred reactive groups for use with viral vaccines, and which will covalently bind to sites on the surface of a virus, include N-hydroxy succinimide (NHS), nitrophenol ester (ONp) and thiazolidine-2-thione (TT) groups.

In the case of electrostatic interaction with the vaccine, charged groups may be incorporated into the polymer chain in order to promote electrostatic attraction to the vaccine.

In one particular example, a recombinant vaccine particle based on an adenovirus vector containing DNA encoding a gene for a target pathogen may be surface-coated with the polymer-adjuvant conjugate in order to increase its ability to stimulate an immune response following expression of the pathogen protein within cells of the recipient. To achieve this the polymer-adjuvant conjugate is produced with a complement of groups that can bind to the surface of the adenovirus vector, linking the polymer to the surface and thereby presenting the adjuvant on the surface of the virus particle. In this embodiment, suitable reactive groups are those capable of producing covalent linkage to the virus (eg NHS, ONP, TT groups) or charged groups.

The binding sites on the vaccine may be naturally occurring or may be introduced. Where binding sites are introduced, these should be complementary to the reactive groups on the polymer-adjuvant construct. For example, a viral vector may be engineered to express specific reactive groups on its surface proteins (for example, free thiol groups), and corresponding reactivity may be introduced into the polymer-adjuvant construct (for example maleimide groups) to enable direct covalent linkage to the virus particle.

When both the polymer-adjuvant construct and the vaccine have multiple complementary reactive groups, it is possible that the product of their linkage together may be an aggregate or even a precipitate. While this may be useful to provide a local depot of adjuvaneted vaccine, the crosslinking effect may be minimised by using one of the components (normally the polymer-adjuvant construct) in excess. Alternatively the polymer-adjuvant construct may be produced with just one residual reactive group, ensuring monovalent linkage to the vaccine. In one embodiment this may be achieved by creating a semitelechelic reactive polymer, where one terminal of each polymer molecule is derivatised with a reactive group and several adjuvantss are incorporated (as derivatised comonomers) into the polymer chain. The terminal reactive group is selected so that it does not react with the adjuvantss, but can be used for linkage of the conjugate to the vaccine.

In an alternative embodiment, the vaccine and adjuvant-polymer construct are present within a single composition, together with a pharmaceutically acceptable carrier or diluent. In a further alternative embodiment, the vaccine and adjuvant-polymer construct are separately formulated to provide two separate compositions. In this latter case, the two compositions may be administered to a patient simultaneously or separately.

The present invention therefore provides compositions comprising the adjuvant-polymer construct or vaccine conjugate of the invention together with a pharmaceutically acceptable carrier or diluent and optionally together with a vaccine. Preferred compositions are free of contamination from micro-organisms and pyrogens.

The compositions of the invention may be formulated for administration in a variety of dosage forms. Thus, they can be administered orally, for example as aqueous or oily suspensions. The compositions of the invention may be formulated for administration parenterally, either subcutaneously, intravenously, intramuscularly, intrasternally, intraperitoneally, intradermally, transdermally or by infusion techniques. Dermal, and intramuscular administration is preferred. The compositions of the invention may be formulated for administration by inhalation in the form of an aerosol via an inhaler or nebuliser.

The formulations for oral administration, for example, may contain, together with the active ingredients mentioned above, solubilising agents, e.g. cyclodextrins or modified cyclodextrins; diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations.

Liquid dispersions for oral administration may be solutions, syrups, emulsions and suspensions. The solutions may contain solubilising agents e.g. cyclodextrins or modified cyclodextrins. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol; solubilising agents, e.g. cyclodextrins or modified cyclodextrins, and if desired, a suitable amount of lidocaine hydrochloride.

Solutions for intravenous or infusions may contain as carrier, for example, sterile water and solubilising agents, e.g. cyclodextrins or modified cyclodextrins or preferably they may be in the form of sterile, aqueous, isotonic saline solutions.

A therapeutically effective amount of an adjuvant-polymer construct of the invention is administered to a subject. The multi-valent display of the adjuvant enables the use of lower adjuvant concentrations than have previously been proposed for non-polymer bound adjuvants. The amount of adjuvant administered is therefore equal to or preferably less than the dosage for a corresponding formulation using the same adjuvant but which is not bound to a polymer. The adjuvant-polymer construct of the invention is typically administered to the subject in a non-toxic amount. The vaccine is also administered in a therapeutically effective and non-toxic amount.

The polymer-adjuvant constructs of the invention are useful in the enhancement and stimulation of immune response in a wide range of different areas of medicine. The invention is therefore useful for the treatment or prophylaxis of infectious diseases, cancer and autoimmune diseases and for the treatment of allergy and hypersensitivity.

Examples of infectious diseases include those caused by an agent selected from the group consisting of a virus, a bacterium, a parasite and a fungus.

The viral infectious disease may be seasonal influenza, avian influenza, respiratory syncytial virus, Human Papilloma Virus, viral hepatitis, HIV/AIDS, Herpes simplex, Varicella zoster, Cytomegalovirus, Dengue fever, Ebola hemorrhagic fever, Hand, foot and mouth disease, Lassa fever, Measles, Marburg hemorrhagic fever, Infectious mononucleosis , Epstein-Barr virus, Mumps, Norovirus, Poliomyelitis, Rabies, Rubella, SARS, Smallpox (Variola), West Nile disease, Yellow fever, rotovirus, Japanese encephalitis, Colorado tick fever, common cold, viral encephalitis, viral gastroenteritis, viral meningitis or viral pneumonia.

The bacterial infectious disease may be Bacterial Meningitis, Staphylococcus aureus (including MRSA), Salmonellosis, Shigellosis Campylobacteriosis, Chlamydia, Lyme disease, Pneumococcal pneumonia, Anthrax, Botulism, Brucellosis, Trachoma, Tuberculosis Cat Scratch Disease, Cholera, Diphtheria, Epidemic Typhus, Gonorrhea, Impetigo, Legionellosis, Leprosy, Leptospirosis, Listeriosis, Melioidosis, Nocardiosis, Pertussis, Plague, Psittacosis, Q fever, Rocky Mountain Spotted Fever, Scarlet Fever, Syphilis, Tetanus, Tularemia, Typhoid Fever, Typhus, bacterial Urinary Tract Infection, Chlamydia trachomatis, Heliobacter pylori

The parasitic infectious may be Malaria, Trypanosomiasis, Schistosomiasis, Cysticercosis, Chagas Disease, Giardiasis, Kala-azar, Leishmaniasis, Filariasis, Amebiasis, Ascariasis, Babesiosis, Clonorchiasis, Cryptosporidiosis, Diphyllobothriasis, Dracunculiasis, Echinococcosis, Enterobiasis, Fascioliasis, Fasciolopsiasis, Free-living amebic infection,—Gnathostomiasis, Hymenolepiasis, Isosporiasis, Metagonimiasis, Myiasis, Onchocerciasis, Pediculosis, Pinworm Infection, Scabies, Taeniasis, Toxocariasis, Toxoplasmosis, Trichinellosis, Trichinosis, Trichuriasis, Trichomoniasis.

The fungal infectious disease may be Candidiasis, Aspergillosis, Blastomycosis, Coccidioidomycosis, Cryptococcosis, Histoplasmosis, Tinea pedis.

Examples of cancer include colorectal cancer, non-small cell lung cancer, prostate cancer, breast cancer, pancreatic cancer, ovarian cancer, hepatic cancer, skin cancer, melanoma, gastric cancer, small cell lung cancer, sarcoma, bladder cancer, oesophageal cancer, cervical cancer, endometrial cancer, testis cancer, renal cell cancer, nasopharyngeal cancer, head and neck cancer, thyroid cancer, glioma, astrocytoma, lymphoma, leukaemia, a myeloproliferative disorder, retinoblastoma, an embryonal tumour or a metastatic cancer.

Examples of autoimmune diseases include rheumatoid arthritis, diabetes mellitus, multiple sclerosis, psoriasis, Crohns disease, ankylosing spondylitis, Graves disease, Hashimotos thyroiditis, idiopathic myxedema, Guillain-Barre syndrome, systemic lupus erythemetosis, immune thrombocytopenia purpura, pemphigus vulgaris, fibromyalgia, myasthenia gravis, sarcoidosis, sjogrens syndrome, Kawasaki's Disease, Lou Gehrig's Disease, a demyelinating disease, a haemolytic anaemia, an autoimmune arteritis, an autoimmune colitis, an autoimmune uveitis, an autoimmune myositis, an autoimmune arthritis and an autoimmune hepatitis.

### EXAMPLES

### Example 1

### Synthesis of Compounds for Binding to Soluble Polymers for use as Polymer Enhanced Adjuvants

Synthesis of N-Pam3Cys-(N′-Boc-2,2′-(ethylenedioxy)diethylamine) (1)

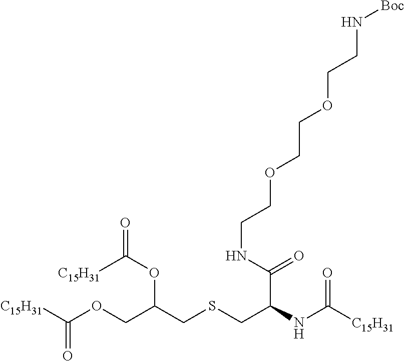

Pam3Cys was modified by carbodiimide coupling to a mono-Boc-protected diamine. Pam3Cys-OH (95 mg, 104 μmol) was dissolved in anhydrous DCM (5 mL) and added to PS-Carbodiimide resin (1.33 mmol/g, 138 mg, 185 μmol) and shaken for 5 min under Ar. N-Boc-2,2′-(ethylenedioxy)diethylamine) (33 mg, 132 μmol) was added in anhydrous DCM (1 ml) and shaking continued for 16 h. TLC (neat EtOAc) found no residual Pam3Cys-OH. The resin was removed by filtration and 50 mg of PS-benzaldehyde resin was added as an amine scavenger. After 24 h shaking the solution was filtered and the solvent removed under reduced pressure. The crude solid was purifed by column chromatography (gradient elution 0-20% MeOH in DCM, product Rf 0.6) and isolated as a white solid.

Synthesis of N-Pam3Cys-(2,2′-(ethylenedioxy)diethylamine) (2)

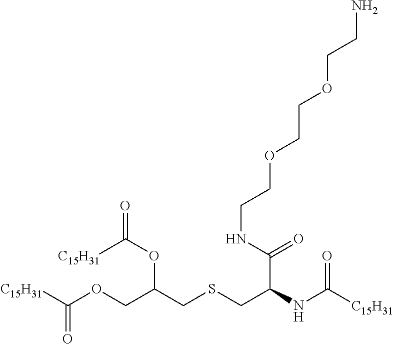

N-Pam3Cys-(N′-Boc-2,2′-(ethylenedioxy)diethylamine) (83 mg, 73 μmol) was dissolved in 1/1 DCM/TFA (4 mL) and stirred gently for 1 h. Solvent and excess acid was removed by azeotroping with toluene and then diethyl ether. The resulting white solid was dissolved in a mixture of DCM and sat. aq. NaHCO3 and stirred vigorously for 2 h. The organic layer was collected and the aqueous extracted with DCM (2×10 mL). The extracts were combined, dried over MgSO4 and evaporated to yield 53 mg of a white solid. MS (ESI+) Found m/z 1041.8 [M+H+] requires 1040.8.

Synthesis of a Ceramide Analogue (3)

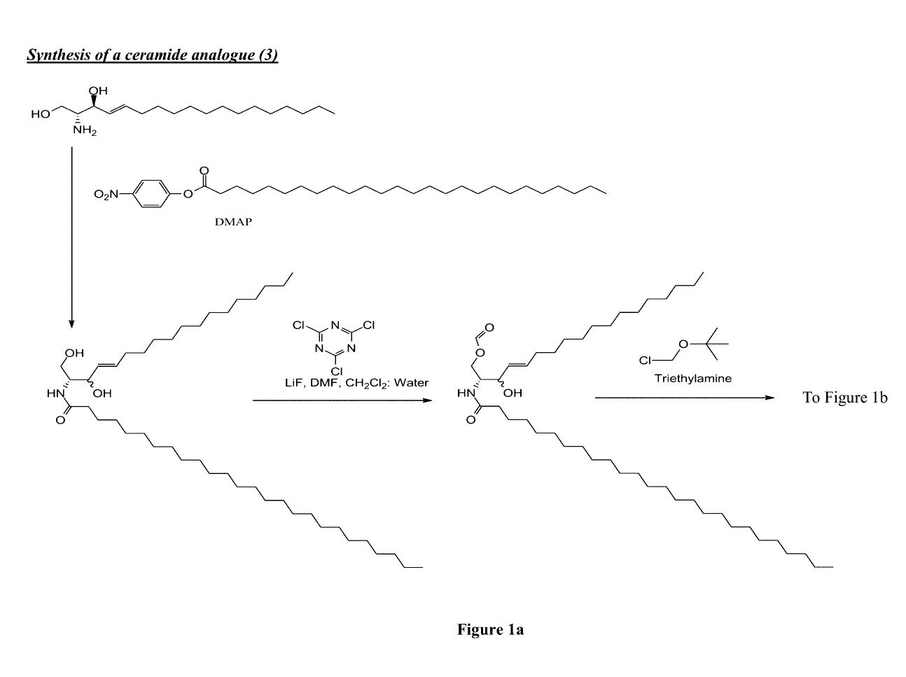

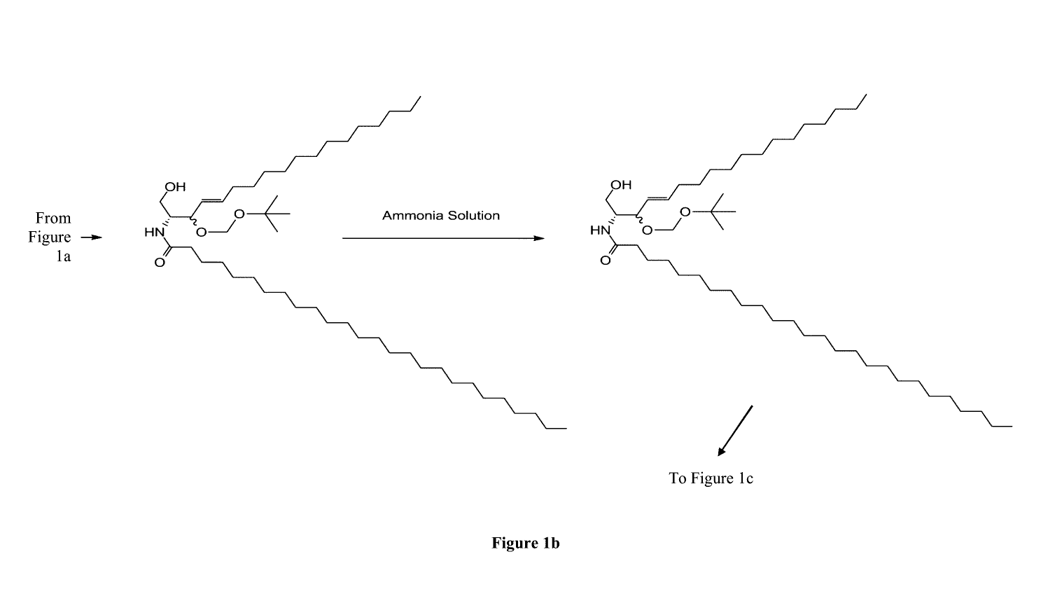

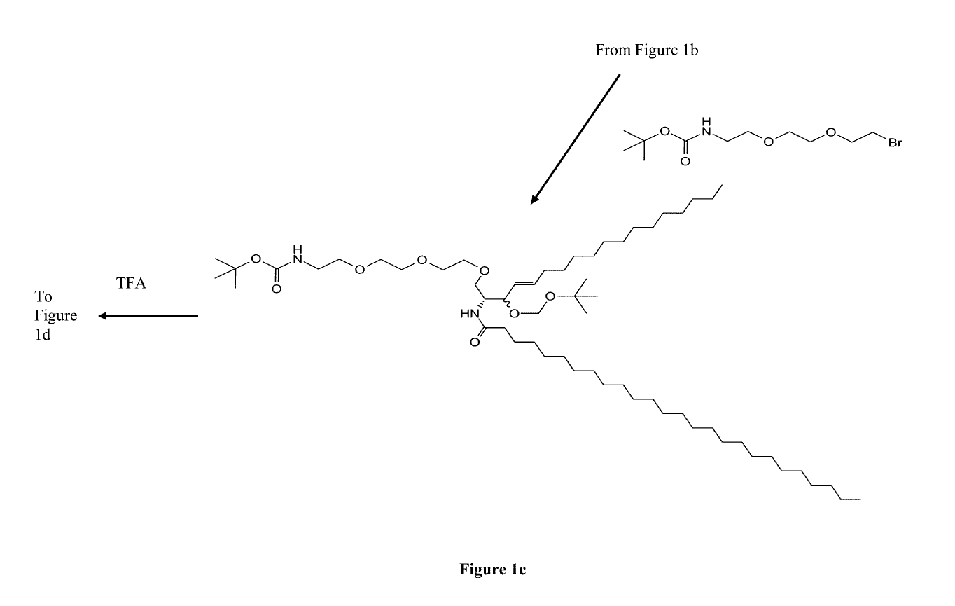

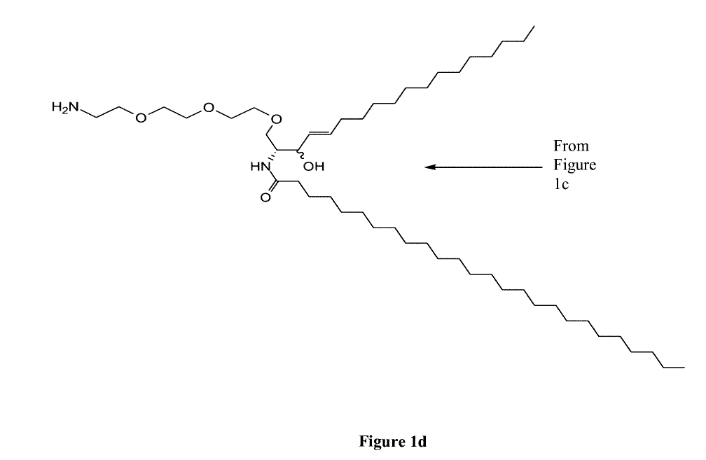

A ceramide analogue was prepared in accordance with the scheme depicted in FIGS. 1a-1d.

### Example 2

### Production of Polymer-Conjugated Adjuvant

Hydrophilic polymers such as poly[N-(2-hydroxypropyl)methacrylamide] bearing multiple pendant amino-, hydroxyl-, or thiol-reactive groups can be modified to bear immunostimulatory molecules such as N-Pam3Cys-(2,2′-(ethylenedioxy) diethylamine) (2) or ceramide analogues such as (3). This example describes the synthesis of poly[HPMA][MA-GG-TT] and its conjugation to ceramide analogue (3).

Synthesis of a Multivalent Aminoreactive Hydrophilic Copolymer:

Synthesis of poly[N-(2-hydroxypropyl)methacrylamide3-(N-methacryloylglycylglycyl) thiazolidine-2-thione]

HPMA (1.00 g, 6.99 mmol), MA-GG-TT (234 mg, 0.77 mmol) and AIBN (200 mg, 1.21 mmol) were dissolved in anhydrous DMSO to a total volume of 10 mL (approximately 12.5 wt % monomer). The solution was deaerated by Ar bubbling for 20 minutes after which the flask was sealed and placed in an oil bath at 60 C with gentle stirring for 6 h. After this time the polymer was precipitated by addition of the solution dropwise to an anhydrous mixture of acetone/ether (3/1). The powder was isolated by centrifugation (15 min. @ 3000 rpm), resuspension in acetone/ether, centrifugation and subsequently dried under vacuum. TT content measured by UV-Vis. spectroscopy in MeOH.

Conjugation of the Amino-Bearing Adjuvant to the Multivalent Aminoreactive Copolymer

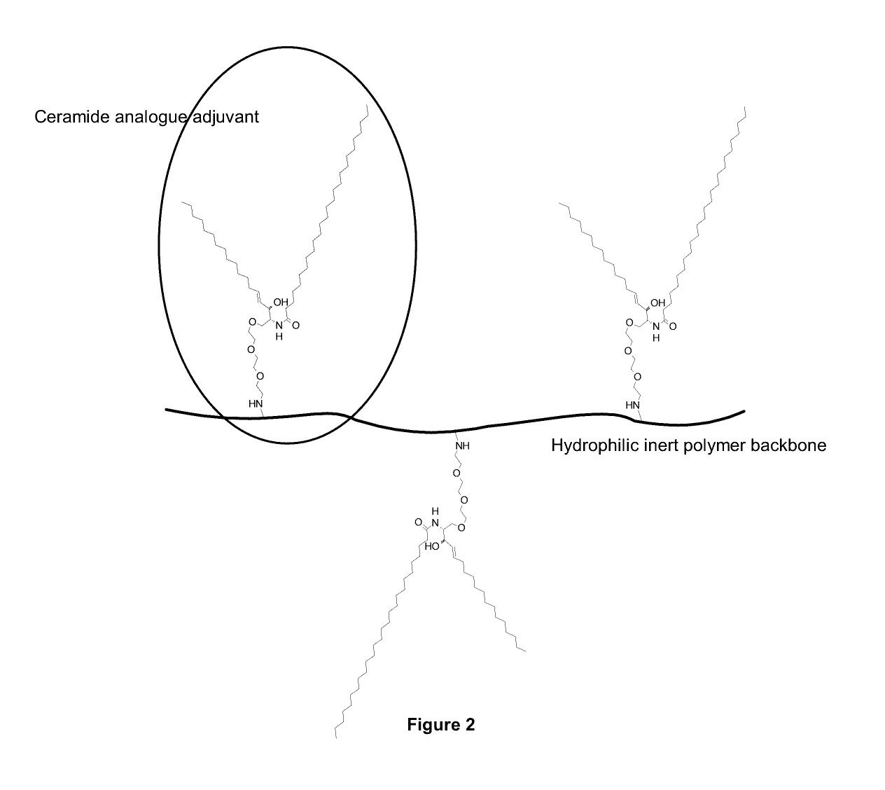

Covalent conjugation of the adjuvant to the copolymer was achieved by mixing in anhydrous dimethyl sulfoxide. The polymer conjugate was then precipitated and dried. Excess reactive groups were removed by hydrolysis and the polymer conjugate was purified by dialysis and lyophilised. The structure of the resulting conjugate is depicted in FIG. 2.

### Example 3

### Linkage of Polymer-Bound Adjuvant to Vaccine

In this example the polymer-bound ceramide derivative is linked to the AMSTTDLEA, a peptide derived from the X protein of hepatitis B virus and known to be recognised by cytotoxic T lymphocytes.

A ceramide-polymer conjugate was synthesised as described in the previous Example, except that reaction conditions and the relative concentrations of reagents were optimised by comparing the effects of reaction time, temperature and concentration of reagents, in order to maintain approx 1 mol % of free reactive groups on the polymers at the time of precipitation. This material was dried and stored.

An oligopeptide with the structure GGGAMSTTDLEA, with blocked carboxy terminus and free amino terminus was produced by cleavage from the solid phase resin. The oligopeptide was dissolved in DMF and allowed to react to completion with the polymer-ceramide conjugate bearing 1 mol % reactive TT groups. This agent was then precipitated, dialysed and stored at −20 degrees.

### Example 4

### Linkage of Positively-Charted Polymer to Ceramide Adjuvant and to Oligopeptide Vaccine via Biodegradable Bonds

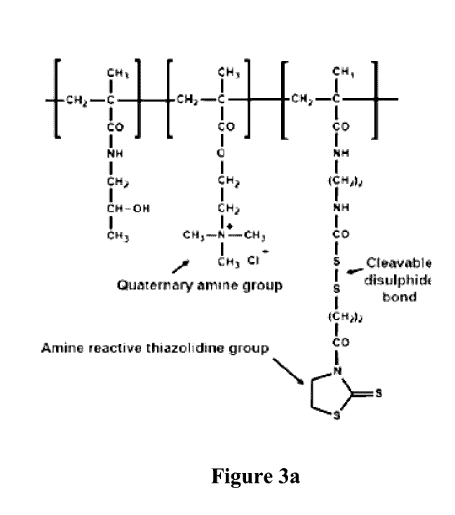

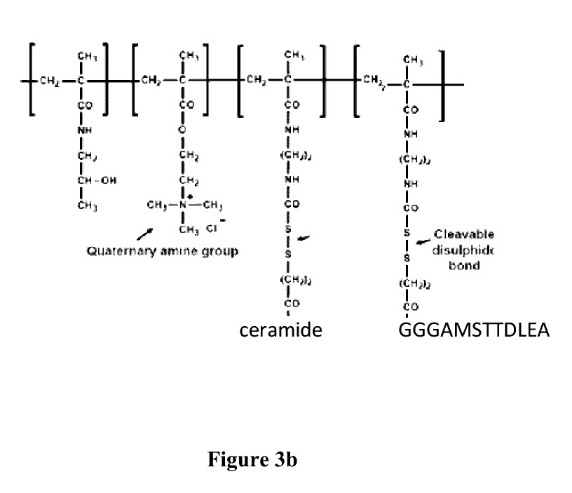

In this example the copolymers based on N-(2-hydroxypropyl)methacrylamide (HPMA) contain monomers bearing quaternary ammonium groups (1.5 mol % in polymerization mixture) and disulphide-bearing side chains terminated in thiazolidine groups (3.4 mol % in product, for reaction with primary amines in the adjuvant and also in the vaccine). The structure of the reactive polymer is shown in FIG. 3a

The reactive polymer was synthesised and characterised as described elsewhere (Subr V, Kostka L, Selby-Milic T, Fisher K, Ulbrich K, et al. (2009) Coating of adenovirus type 5 with polymers containing quaternary amines prevents binding to blood components. J Control Release 135: 152-158.). It had weight average molecular weight 77,200 and number average molecular weight 32,200.

Ceramide was linked as described above, with 1 mol % of thiazolidine groups remaining unreacted to enable subsequent covalent linkage of the peptide antigen. In this example the peptide antigen was derived from the hepatitis virus X antigen, and had the structure: GGGAMSTTDLEA, with blocked carboxy terminus and free amino terminus (see FIG. 3b).

### Example 5

### Preparation of Nanogel-Bound Adjuvant

Nanogel core particles were synthesized by free-radical precipitation polymerization, as previously reported (Blackburn et al., Colloid Polym Sci. 2008; 286(5): 563-569). The use of thermally phase separating polymers enables the use of precipitation polymerization for the synthesis of highly monodispersed nanogels. The molar composition was 98% N-isopropylmethacrylamide (NIPMAm), 2% N,N′-methylenebis(acrylamide) (BIS), with a total monomer concentration of 140 mM. The solution also contained a small amount (about 0.1 mM) of acrylamidofluorescein (AFA) to render the nanogels fluorescent for visualization via confocal microscopy. In a typical synthesis, 100 mL of a filtered, aqueous solution of NIPMAm, BIS, and sodium dodecyl sulfate (SDS, 8 mM total concentration) was added to the reaction flask, which was then heated to 70° C. The solution was purged with N₂ gas and stirred vigorously until the temperature remained stable. The AFA was added, and after 10 min the reaction was initiated by the addition of a 1 mL solution of 800 mM ammonium persulfate (APS) to make the final concentration of APS in the reaction ˜8 mM. The solution turned turbid, indicating successful initiation. The reaction was allowed to continue for 4 h under an N2 blanket. After synthesis, the solution was filtered through Whatman filter paper to remove a small amount of coagulum.

10 mL of the core nanogel solution and 0.0577 g of SDS were first added to a three-neck round-bottom flask and heated under N2 gas to 70° C. A 50 mM monomer solution with molar ratios of 97.5% NIPMAm, 2% BIS, and 0.5% N-glycyl methacrylamide was prepared in 39.5 mL of dH2O. The solution was added to the three-neck round-bottom flask, and the temperature was stabilized at 70° C. while continuously stirring. The reaction was initiated by a 0.5 mL aliquot of 0.05 M APS. The reaction proceeded for 4 h under N2 gas. Following the synthesis, the solution was filtered through Whatman filter paper, and the nanogels were purified by centrifugation followed by resuspension in dH2O.

Conjugation of an Amine Bound Adjuvant to the Nanogel Core

The acid functionalized nanogels were conjugated to the amine bearing adjuvant firstly by activation of the acid functionality with N-hydroxysuccinimide using dicyclohexylcarbodiimide. Following purification, the addition of the amine bearing adjuvant reacts directly with the nanogel surface.

### Example 6

### In Vitro Activity Assessment Using NfkB-Luciferase Reporter Cells of the TLR2 agonist Pam3Cys Conjugated to HPMA Copolymers

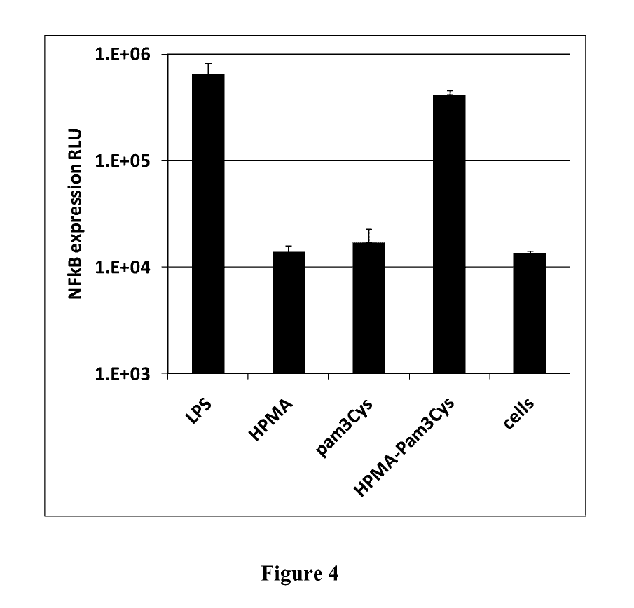

Adjuvant activity of a polymer-Pam3cys conjugate was assessed in vitro using THP-1 cells transfected with a plasmid containing luciferase under the control of the NfkB promoter (U937-luc). U937-luc cells were grown to a density of 1e6 per ml before exposure to 100 ng/ml of LPS, 100 ng/ml Pam3Cys, 100 ng/ml pHPMA or 100 ng/ml pHPMA-Pam3Cys. After 8 hours cells were pelleted, lysed and evaluated for luciferase expression (FIG. 4). In this study the HPMA copolymer used had a molecular weight average of approximately 20 kDa and contained 5.22 wt % Pam3cys (GCMS). The HPMA was bound to Pam3cys by a glycine-glycine spacer. The polymer-bound adjuvant was prepared in accordance with the techniques described in Examples 1 and 2. The data shows that the luciferase signal from 100 ng of Pam3Cys-HPMA was 24.1 fold higher than Pam3Cys alone, even though only 5.22 ng of this material was Pam3Cys. The specific activity of Pam3Cys per molecule, when presented by the polymer is 24.1×19.2=462 fold higher.

### Example 7

### In Vitro Activity Assessment of the TLR2 Agonist Pam2Cys Conjugated to HPMA using Murine Bone Marrow Derived Dendritic Cells (BMDCs)

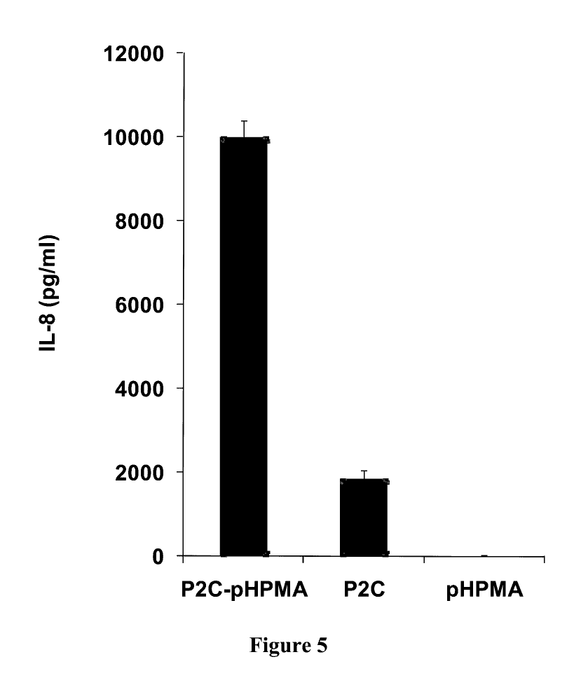

BMDCs are known to respond to TLR2 agonists resulting in expression of inflammatory cytokines including IL-8. We used this model to demonstrate the potency of Pam2Cys when linked to HPMA. In this example the polymer was approximately 80 kDa and was prepared with 5 wt % Pam2Cys. The HPMA was bound to Pam2Cys by a glycine-glycine spacer. The polymer-bound adjuvant was prepared in accordance with the techniques described in Examples 1 and 2. BMDCs were exposed to 50 ng/ml Pam2Cys or polymer bound Pam2Cys for 24 hours. After which the supernatant was collected and IL-8 expression determined by ELISA. FIG. 5 shows that multiple presentation of Pam2Cys on HPMA results in higher levels of dendritic cell activation with 20 fold lower quantity of ligand relative to Pam2Cys on its own.

### Example 8

### Vaccine Conjugate consisting of Antigenic Peptide (influenza M2e) and TLR Agonist (Pam3Cys) Displayed along Polymer (HPMA) Backbone

Influenza M2e peptide (SLLTEVETPIRNEWGCRCNDSSD) is a surface antigen highly conserved across different strains of virus. Although poorly immunogenic on its own, M2e is often co-administered with adjuvants. In this example multivalent polyHPMA bearing 5 wt % Pam3cys and 1 mol % free reactive TT groups on pendant GSGS side chains was reacted to completion (in DMSO) with the free amino terminus of the oligopeptide. Free oligopeptide was removed by column chromatography.

1-17. (canceled)

18. An adjuvant-polymer construct comprising 3 or more adjuvants in a multivalent display, each adjuvant covalently linked to a linear polymer backbone, wherein the backbone comprises poly amino acids.

19. The adjuvant-polymer construct of claim 18, wherein the backbone comprising poly amino acids is synthetic.

20. The adjuvant-polymer construct of claim 18, wherein the adjuvants are directly linked to the linear polymer backbone.

21. The adjuvant-polymer construct of claim 18, wherein the adjuvants are linked to the linear polymer backbone via a spacer.

22. The adjuvant-polymer construct of claim 21, wherein the spacer is a PEG spacer.

23. The adjuvant-polymer construct of claim 21, wherein the spacer is a butylene spacer.

24. The adjuvant-polymer construct of claim 21, wherein the spacer is an aryl spacer.

25. The adjuvant-polymer construct of claim 18, wherein 10 to 50 adjuvants are covalently linked to the linear polymer backbone.

26. The adjuvant-polymer construct of claim 18, wherein at least 5 adjuvants are covalently linked to the linear polymer backbone.

27. The adjuvant-polymer construct of claim 18, wherein 3-5 adjuvants are covalently linked to the linear polymer backbone.

28. The adjuvant-polymer construct of claim 18, wherein the adjuvant molecules are covalently attached to the linear polymer backbone via an amide linkage.

29. The adjuvant-polymer construct of claim 18, wherein each of the adjuvant molecules are the same.

30. The adjuvant-polymer construct of claim 18, wherein each of the adjuvant molecules are different.

31. The adjuvant-polymer construct of claim 18, wherein the weight average molecular weight of the polymer is 1 to 100 kDa.

32. An adjuvant-polymer construct of claim 18, wherein the weight average molecular weight of the linear polymer backbone is from 5 to 40 kDa.

33. An adjuvant-polymer construct of claim 18, wherein the weight average molecular weight of the linear polymer backbone is from 1 to 5 kDa.

34. The adjuvant-polymer construct of claim 18, wherein a vaccine is conjugated to the adjuvant-polymer construct.

35. The adjuvant-polymer construct of claim 18, wherein each adjuvant is attached as a pendant side chain.

36. The adjuvant-polymer construct of claim 34, wherein the vaccine is covalently attached to the adjuvant-polymer construct.

37. The adjuvant-polymer construct of claim 34, wherein the vaccine comprises peptides.

38. The adjuvant-polymer construct of claim 34, wherein the vaccine comprises a protein.

39. The adjuvant-polymer construct of claim 34, wherein the vaccine is directed to cancer-associated antigens.

40. The adjuvant-polymer construct of claim 18, wherein the adjuvants are selected from the group consisting of: lipoglycans, lipopolysaccharide, lipoteichoic acid, peptidoglycan, synthetic lipoproteins, zymosan, glycolipids, polyinosine-polycytidylic acid, monophosphoryl Lipid A, flagellin, imidazoquinoline-compounds, guanosine, tumor necrosis factor alpha (TNFa) or peptides thereof, interleukin 2 (IL-2), interleukin 4 (IL-4), interleukin 8 (IL-8), CD40 molecule, tumor necrosis factor receptor superfamily member 5 (CD40), tumor necrosis factor receptor superfamily member 4 (OX40), colony stimulating factor 2 (granulocyte-macrophage) (GM-CSF), and cytosine-phosphate-guanine-containing (CpG-containing) sequences in bacterial DNA or synthetic oligonucleotides.

41. The adjuvant-polymer construct of claim 18, wherein the adjuvants bind Toll-like receptors.

42. The adjuvant-polymer construct of claim 41, wherein the Toll-like receptor is selected from TLR1, TLR2, TLR3, TLR4, TLR5, TLR6, TLR7, TLR8, or TLR9.

43. The adjuvant polymer construct of claim 42, wherein the Toll-like receptor is TLR7 or TLR8.

44. The adjuvant polymer construct of claim 42, wherein the Toll-like receptor is TLR7 and TLR8.

45. The adjuvant-polymer construct of claim 43, wherein a vaccine is conjugated to the adjuvant-polymer construct.

46. The adjuvant-polymer construct of claim 45, wherein the vaccine is covalently attached to the adjuvant-polymer construct.

47. The adjuvant-polymer construct of claim 45, wherein the vaccine comprises peptides.

48. The adjuvant-polymer construct of claim 45, wherein the vaccine comprises a protein.

49. The adjuvant-polymer construct of claim 45, wherein the vaccine is directed to cancer-associated antigens.

50. A composition comprising the adjuvant-polymer construct of claim 18 and a pharmaceutically acceptable carrier or diluent.

51. A composition comprising the adjuvant-polymer construct of claim 39 and a pharmaceutically acceptable carrier or diluent.

52. A composition comprising the adjuvant-polymer construct of claim 43 and a pharmaceutically acceptable carrier or diluent.

53. A composition comprising the adjuvant-polymer construct of claim 49 and a pharmaceutically acceptable carrier or diluent.

54. A method for stimulating or enhancing an immune response in a subject in need thereof, comprising administering to said subject an effective, non-toxic amount of (a) an adjuvant-polymer construct as defined in claim 18 or (b) a composition comprising the adjuvant-polymer construct as defined in claim 18 and pharmaceutically acceptable carrier or diluent, wherein when the adjuvant-polymer construct or composition does not comprise a vaccine, the method further comprises a further step of administering to said patient an effective and non-toxic amount of a vaccine.

55. The method of claim 54, wherein the subject suffers from cancer.

56. The method of claim 55, wherein the cancer is selected from the group consisting of: colorectal cancer, non-small cell lung cancer, prostate cancer, breast cancer, pancreatic cancer, ovarian cancer, hepatic cancer, skin cancer, melanoma, gastric cancer, small cell lung cancer, sarcoma, bladder cancer, oesophageal cancer, cervical cancer, endometrial cancer, testis cancer, renal cell cancer, nasopharyngeal cancer, head and neck cancer, thyroid cancer, glioma, astrocytoma, lymphoma, leukaemia, a myeloproliferative disorder, retinoblastoma, an embryonal tumour and a metastatic cancer.
